(19) United States
(12) Patent Application Publication
(10) Pub. No.: US 2020/0226495 A1
(43) Pub. Date: Jul. 16, 2020

(54) METHOD, SYSTEM AND APPARATUS USING FIELD LEARNING TO UPGRADE TRENDING SENSOR CURVES INTO FUEL GAUGE BASED VISUALIZATION OF PREDICTIVE MAINTENANCE BY USER DRIVEN FEEDBACK MECHANISM

(71) Applicant: MachineSense, LLC, Baltimore, MD (US)

(72) Inventor: Biplab PAL, Ellicott City, MD (US)

(73) Assignee: MachineSense, LLC, Baltimore, MD (US)

(21) Appl. No.: 16/828,257

(22) Filed: Mar. 24, 2020

Related U.S. Application Data
(63) Continuation of application No. 15/385,295, filed on Dec. 20, 2016, now abandoned.
(60) Provisional application No. 62/299,996, filed on Feb. 25, 2016.

(51) Int. Cl. G06N 20/00; G06N 5/04; G05B 23/02; G06F 30/20

(52) U.S. Cl. CPC G06N 20/00; G06N 5/045; G16H 40/40; G06F 30/20; G06N 5/04; G05B 23/0283

(57) ABSTRACT

A field learning system comprising a system of feedback using a user interface in a web based and mobile application to overcome the difficulty and infeasibility of supervised machine learning systems used for modeling failure states of machines.

### CROSS REFERENCE TO RELATED PATENT APPLICATION

This patent application is a 35 USC 120 continuation of co-pending U.S. patent application Ser. No. 15/385,295 filed 20 Dec. 2016. The '295 application was published as United States patent publication 2017-0178030 on 22 Jun. 2017.

The '295 application claims the priority under 35 USC 120 of U.S. provisional application Ser. No. 62/269,996 filed 20 Dec. 2015 and entitled “Field Learning System to Upgrade Trending Sensor Curves into Fuel Gauge Based Visualization of Predictive Maintenance by User Driven Feedback Mechanism.”

### STATEMENT REGARDING FEDERALLY-SPONSORED RESEARCH AND FUNDING SUPPORT

Not applicable.

### INCORPORATION BY REFERENCE

Applicant hereby incorporates by reference the disclosures of pending U.S. Ser. No. 14/599,461 filed 17 Jan. 2015, published as U.S. patent publication 2016/0209381 A1 on 21 Jul. 2016; U.S. Ser. No. 14/628,322 filed 23 Feb. 2015, published as U.S. patent publication 2016/0245279 A1 2016 on 25 Aug. 2016; PCT/US2016/018820 filed 20 Feb. 2016, published as World Intellectual Property Organization publication 2016/137848 A1 1 Sep. 2016; U.S. Ser. No. 14/790,084 filed 2 Jul. 2015, published as U.S. patent publication 2016/313261 A1 on 27 Oct. 2016; PCT/US2016/028724 filed 22 Apr. 2016, published as World Intellectual Property Organization publication 2016/176111 on 3 Nov. 2016; U.S. Ser. No. 14/726,696 filed 1 Jun. 2016, published 1 Dec. 2016 as U.S. patent publication 2016/0349305 A1; U.S. Ser. No. 14/934,179 filed 6 Nov. 2015, published on 6 Oct. 2016 as U.S. patent publication 2016/0291552 A1; PCT/US2015/066547 filed 18 Dec. 2015, published 26 May 2016 as World Intellectual Property Organization publication 2016/081954; U.S. Ser. No. 14/977,675 filed 22 Dec. 2015, published on 25 Aug. 2016 as U.S. patent publication 2016/0245686 A1; PCT/US2016/18831 filed 21 Feb. 2016, published on 1 Sep. 2016 as World Intellectual Property publication 2016/137,849 A2; U.S. Ser. No. 15/049,098 filed 21 Feb. 2016, published on 25 Aug. 2016 as U.S. patent publication 2016/0245765 A1.

### BACKGROUND OF THE INVENTION AND SUMMARY OF THE PRIOR ART

Machine condition based monitoring is growing in use to reduce downtime of machines resulting from unplanned breakdowns. See, for example, Jaouher Ben Ali, Nader Fnaiech, Lotfi Saidi, Brigitte Chebel-Morello, Farhat Fnaiech, Application of empirical mode decomposition and artificial neural network for automatic bearing fault diagnosis based on vibration signals, Applied Acoustics, 20 Sep. 2014.

To predict machine failures well in advance so that the machine operators can take adequate steps in advance of any failure to repair the relevant machine, predictive models of the failure modes of the machines are required. Heretofore such predictive models have been built from time series data using appropriate sensor technology for a given physical parameter. For vibration see, for example, C. Lu, J. A. Stankovic, G. Tao, Design and Evaluation of a Feedback Control EDF Scheduling Algorithm; Transactions of the IEEE, 3 Dec. 1999. For sound see, for example, Widodo A., Yang B., Support Vector Machine in Machine Condition Monitoring and Fault Diagnosis, 2007. For infrared temperature see, for example, P. K. Kankar, Satish C. Sharma, S. P. Harsha, Fault Diagnosis of Ball Bearings Using Machine Learning Methods, Expert Systems with Applications, Volume 38, Issue 3, Mar. 2011, Pages 1876-1886.

Known conventional systems for automated detection of machine failure states depend on supervised learning. See, for example, Polycarpou et al., Automated Fault Detection and Accommodation: a Learning Systems Approach, IEEE Transactions on Systems, Management, and Cybernetics, 1995.

In such systems, a “training file” is required containing prior data regarding operating and failure states of the relevant machine. However, “training” data needed to construct such a “training file” for machine failure modes is not, as a practical matter, available in a wide range of situations such as where the physical parameter sensor has to be mounted on a machine already deployed and operating in a factory, or where the machine has already failed. Such a machine being used in day to day production on a factory assembly line or elsewhere cannot be driven to failure solely for the purpose of obtaining data for a “training file”.

Using data from one machine to predict failure of another machine is not the answer as respecting obtaining reliable, useful data for predicting failure. Even if machine models are the same, an older machine may not have same failure mode(s) as that of a new machine of the same model.

U.S. Pat. No. 5,691,707 deals with the problem of monitoring bearing performance in machines having one or more apertures sized and configured for grease fittings for lubricating the bearings. The '707 patent discloses sensors for temperature and vibration to detect bearing failure but the '707 patent does not disclose use of feedback to reinforce the failure model developed therein for better accuracy of failure detection procedures.

U.S. Pat. 4,453,407 discloses vibration diagnosis and associated apparatus for rotary machines. The '407 patent approach is capable of discriminating causes of the sensed vibration due to unbalanced mass. The '407 patent also discloses a method and apparatus for automatically discriminating whether unbalanced vibration is attributable to abrupt mass unbalance or to thermal bow. However, the '407 patent does not disclose using feedback from a machine operator or otherwise to improve the failure mode model where the model may not be working well.

U.S. Pat. No. 7,308,322 discloses control systems and methodologies for controlling and diagnosing the health of a motorized system and/or components thereof. Diagnosis of the system or component health is accomplished using advanced analytical techniques such as neural networks, expert systems, data fusion, spectral analysis, and the like, wherein one or more faults or adverse conditions associated with the system may be detected, diagnosed, and/or predicted. However, the disclosed method is based on supervised learning and it does not address the problem of using such a learning system on older or already deployed machines.

U.S. patent publication 2006/0095230A1 discloses a system and method for improving diagnostic aids such as fault trees and repair manuals using feedback in the form of repair data from a distributed base of data collection devices used by technicians. Although the disclosed method uses a feedback system operated by a technician, corrective actions are done manually and models are not updated via an automated algorithm of multiple data extractions.

### SUMMARY OF THE INVENTION

In one of its aspects, this invention solves the problem of requiring prior “training data” for supervised learning failure state analysis by applying physics and statistics based models, which are universally validated, for subassemblies of a machine. Physics based and statistically based models in general are based on parametric formulae that do not require any machine failure mode data for their formulation since these models are based on laws of classical mechanics. However, because applicability and reliability of such models may be limited due to economic limitations, uncontrollable or unanticipated physical parameters, and variations of the same, such as thick gearboxes which do not transfer vibration to a sensor effectively, a system of feedback, as presented in FIG. 1, is used to augment reliability and accuracy.

In another one of its aspects, this invention provides a method of predicting machine failure where the method proceeds by connecting a physical parameter sensor, or more than one physical parameter sensor, to the machine of interest. Suitable parameters include sound, vibration and other physical parameters having measurable characteristics. Among the measurable characteristics that may be measured by the sensors are: amplitude, frequency, relative humidity, velocity, revolutions per minute, skewness/eccentricity of a rotating member, voltage, current, phase, inductance, impedance, capacitance, surface temperature, infrared temperature, air temperature, and the like. The method then collects data respecting the selected physical parameter(s) during acceptable and unacceptable machine operation. The method then proceeds by segregating the data collected during acceptable machine operation from data collected during unacceptable machine operation. The method proceeds with determining at least one statistical distribution of the acceptable and unacceptable machine operating data. Next, the difference in the acceptable and unacceptable machine operation data are determined as respecting a selected characteristic of the collected data. Finally, time to machine failure is computed as a function of the physical parameter(s) based on the determined difference in the acceptable and unacceptable operation data.

In yet another one of its aspects, this invention provides a method for maintaining a physical parametric mathematical model of a machine subassembly of interest. The method commences by selecting the physical parameter(s) of interest as respecting the machine subassembly. The method next proceeds by connecting sensors for the selected parameters to an embodiment of the machine subassembly. Next, data are collected from the sensors from machine operation. This data are transferred to a cloud-based database. A physics and statistics based universally validated model for the subassembly of interest is executed using the collected data to produce a result. If the result is an accepted improvement in the physical parametric mathematical model of the machine subassembly of interest, the model is replaced according to the improvement. However, if the result is unacceptable improvement or perhaps a decline, the method proceeds by modifying the model and repeating the steps of collecting data, transferring the collected data, executing the model with the newly-collected data, and checking the result.

In still another aspect of the invention, there is provided a method for predicting machine failure, where the method commences by selecting a subassembly of the machine. The method then selects a universally validated physics and statistically based mathematical model of the selected subassembly. The method then selects a physical parameter or perhaps several physical parameters from the model for analysis as respecting machine failure. The method then proceeds by connecting a sensor for each selected physical parameter to the selected subassembly so the sensor is in operative disposition with the selected subassembly to collect data therefrom. The machine is then started and data are collected from the sensor at two different times during machine operation. The method proceeds by extracting data points for one or more characteristics of the physical parameter(s) of interest from the collected data. The method further proceeds by determining whether the extremes of the extracted data points for the selected characteristic of the physical parameter are separated by pre-selected criteria. If the extremes of the extracted data points for the selected characteristic are separated by at least the pre-selected criteria, the method proceeds with executing an algorithm processing the extracted data points from one extreme to predict machine failure.

In the course of practice of this aspect of the invention, data collected from the sensors during machine operation is preferably dynamically transmitted to a data hub, preferably using a portable or personal electronic device such as a cell phone or a tablet, and thereafter transferred from the data hub to a cloud-resident database for storage therein.

In the practice of this aspect of the invention, the pre-selected separation criterion for data analysis is preferably six sigma.

The method of the invention yet further includes checking the predicted machine failure results and if unsatisfactory, providing indicia thereof to the cloud-resident database for use in updating the selected model. Most desirably, the indicia provided to the cloud-resident database are provided using a mobile electronic device which transmits results of the unsatisfactory failure result prediction to the cloud-resident database for further processing by an algorithm resident therein.

In one embodiment of this invention as presented in FIG. 1, vibrational data are collected in time series and then used to extract various “features” of the sensed vibration. “Features” or characteristics can also be extracted for time series data of magnetic fields, temperature, etc. Time series data representing various failure states can be used for modeling only if two contrasting states of the machine are separated by six-sigma (six standard deviations separates the mean and the accepted extreme where the mean would represent the parameter value during normal accepted machine operation and the extreme would represent the parameter value upon occurrence of a failure) for at least one characteristic, out of the many characteristics of vibration, that has been extracted as depicted in FIG. 2. In the flowchart presented as FIG. 2, multiple parameters are qualified for six sigma criteria and best parameter selection is done using an algorithm as presented in FIG. 2. If no six-sigma separation is applicable, multi parameter classification is done using an SVM/Neural Network/OR Logic Engine. Feedback is then again collected and algorithms are updated with respect to feedback achieved to improve performance, as depicted in FIG. 3.

While the foregoing summarizes the invention and the manner of practicing it in a manner that one of skill in the art can practice the invention, it is to be understood that the foregoing summary of the invention is only a summary and that the invention has aspects broader than those recited. The invention may be implemented in embodiments other than those disclosed herein and may be practiced using apparatus other than that disclosed herein. It is further to be understood that the drawings are attached for purposes of explanation only and that one of skill in the art, upon reading the foregoing description and summary of the invention and looking at the drawings, might contemplate alternate means of practice of the invention. All of such alternate means are deemed to be within the scope of the invention so long as those alternate means achieve essentially the same result in essentially the same way as the invention and are functionally related to the function of this invention.

### DESCRIPTION OF THE DRAWINGS

FIG. 1 is a graphical presentation of the invention and operation thereof.

FIG. 2 is a flow chart illustrating the feedback algorithm portion of the invention in one exemplary embodiment.

FIG. 3 is a graphical presentation of the predictive maintenance aspect of the invention using a feedback mechanism.

FIG. 4 is a simulated screen shot of a blower gauge for an oil check in accordance with one example set forth in the specification below.

FIG. 5 is a plot of a distribution of a blower parameter in the normal state when the blower is being checked for vibration.

FIG. 6 is a plot of a distribution of a blower parameter in the failure state when the blower is being checked for vibration.

FIG. 7 is a plot of average value of harmonics as a function of the order of the harmonies for the rotor of an electric motor.

### DETAILED DESCRIPTION OF THE INVENTION

As used herein, the following terms shall have the meanings stated:

“Gauge”: A visual representation by means of which the current condition of various subassemblies of a machine is displayed. The main purpose of having a gauge for different subassemblies (such as, a blower, heater, etc.) of a machine is to predict the degraded state of the machine by using appropriate color coding (such as red, green, yellow) alerting the targeted recipients in advance so that adequate response time to recheck or repair the machine is available.

“Machine”: A collection of any number of subassemblies, each of which is connected to or in connection with at least one of the other subassemblies to produce a desired result.

“Physics and statistics based models” mean parametric mathematical models in the form of one or more formulae, based on the laws of classical mechanics.

When models are said to be “universally validated”, this denotes that the model has been so widely used and so successfully used that the validity of the model cannot be reasonably be questioned.

Physics based models of most widely used subassemblies are well known.

This invention applies to those machines consisting of combinations of well-known subassemblies (there can be multiple such subassemblies in a machine). For a known subassembly, machine wearable sensors of the type required to generate the machine health data are also assigned from a rule database such as that given below:

| Subassembly | Sensor Physical Parameter |
|---|---|
| Blower | Vibration, Vacuum |
| Motor | Vibration, Power Factor, Current |
| Actuator | Magnetic Field, Vacuum |
| Belt | Vibration |
| Gearbox | Vibration, Power Factor, Current |
| Cutter | Vibration, Power Factor, Current |
| Heater | Power Factor, Current |

In order for physics based models to work, every subassembly is assumed to have a known set of issues which are most frequently encountered. Although variation of design of subassemblies can lead to a higher number of issues, this invention is primarily concerned with the most frequently occurring issues, which are tabulated in Table-1:

TABLE 1
Predictive Maintenance Gauges for Different Subassemblies

| | Bearing | Heater | Actuator | Rotor | Abusive |
|---|---|---|---|---|---|
| | Failure | Failure | Failure | Balance | Operation |
| Motor | R/Y/G | x | x | R/G | Yes |
| Gearbox | R/Y/G | x | x | x | Yes |
| Heater | x | R/Y/G | x | x | Yes |
| Actuator | x | x | R/G | x | x |
| Belt | x | x | x | x | Yes |
| Tension | | | | | |
| Blower | R/Y/G | x | R/G | x | Yes |

Color Code:
Red (R) = Already Failed, Yellow (Y) = Approaching Failure, Green (G) = Healthy State

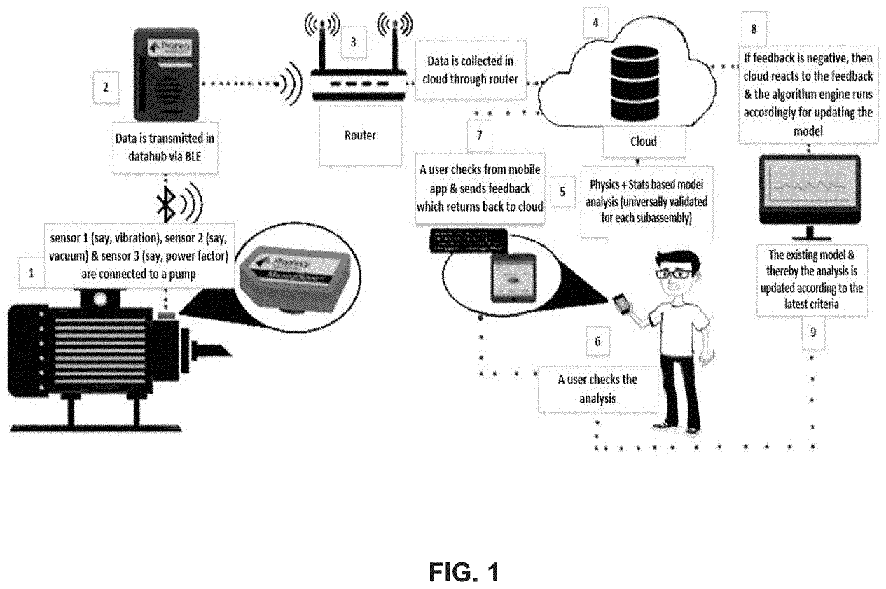

In one preferred practice of the invention, data flows from a parameter sensor either to a cloud-based server or to a local server. The server hosts an algorithm engine which delivers relevant machine conditions to a mobile application device, such as a cell phone, tablet, or the like. FIG. 1 illustrates this data flow.

In this one of its aspects, the invention utilizes sensors for various different physical parameters such as vacuum, vibration, power factor, and current. The sensors, one of which there may be only one or more than one or there may be more than one, are mounted on a machine. For example, in the case of a vibration sensor, vibration data are captured by mounting the vibration sensor on a selected surface of the machine.

Numerical data obtained from the sensors are transmitted to a datahub, for example in a Raspberry Pi, using BLUETOOTH or any other suitable wireless connection protocol.

The data are then most preferably sent wirelessly via the Internet to a selected cloud storage device using a router.

The invention then proceeds with physics and statistics based models from these data for predictive maintenance analysis; the models have been universally validated for subassemblies of the machine.

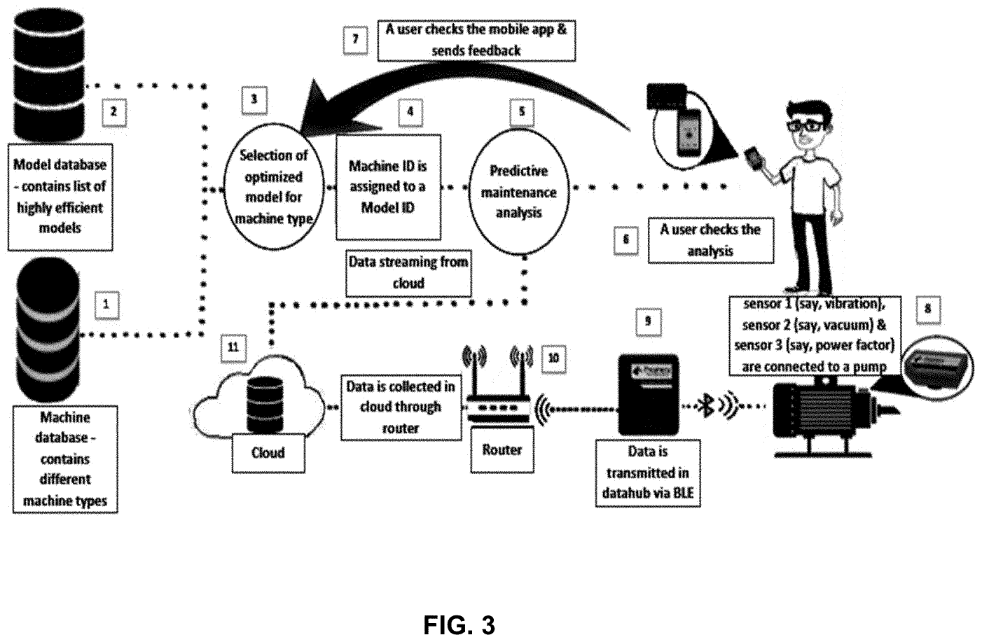

Alerts based on predictive maintenance analysis, as depicted schematically in FIG. 3, are then sent to a user in order to warn of possible failure. A user receives the alerts on a mobile device such as a smart cellphone or a tablet and sends feedback which is again preferably stored in the cloud.

If the feedback is negative, namely if the user is dissatisfied with the analysis, the algorithm reacts to the feedback and the algorithm engine updates the model.

But if the feedback is positive, namely the user is satisfied with the analysis, then the procedure may be continually or periodically repeated as necessary respecting the machine of interest.

After acceptance by the user as being satisfactory, the most recent model is saved in the database as the working model.

The feedback algorithm utilizes the feedback obtained from a user to optimize the predictive models. These optimized physics and statistics based models are then used in the course of performing predictive maintenance of various machines, computers, or assemblies, in various states of operation.

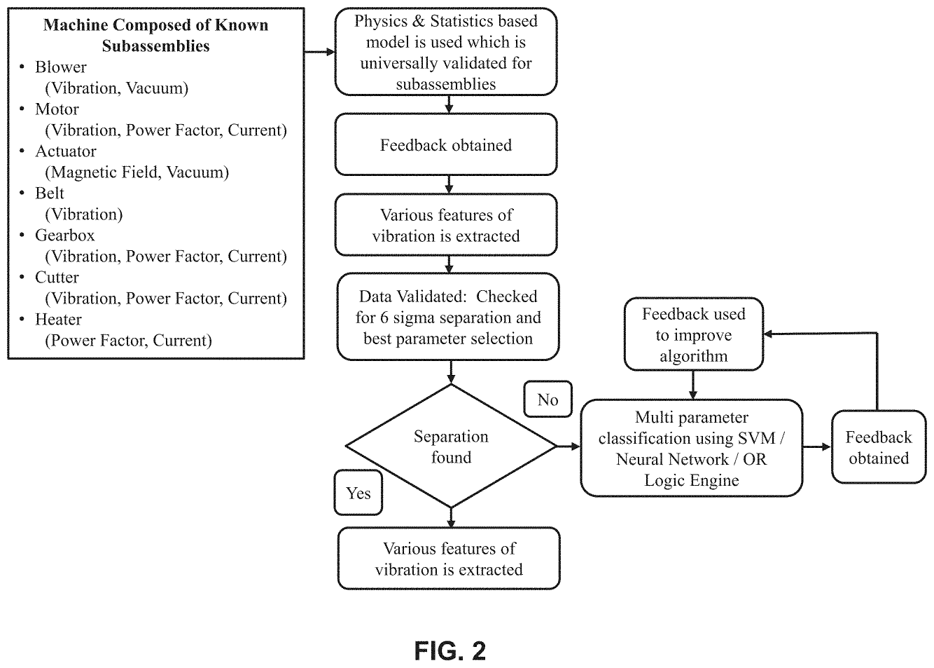

Referring to FIG. 2, in order to detect the good and bad states, time series data are smoothed and the maxima and minima are detected. A “good state” is detected as one of these extrema. For example, in the case of a vibration sensor, data characteristics based on amplitude of vibration and azimuthal angles are extracted and checked for the reliability of data.

If reliability is achieved, then the gauges are activated automatically. If a satisfactory result is not obtained, the system informs the user that failure state classification is not possible; in other words, predictive failure analysis for the machine and the select physical parameters cannot be performed.

For the algorithm, a model database and a machine database are used which are as follows:

The machine database is a database of known machine types.

The model database is a database of highly efficient physics and statistics based models which are universally validated for subassemblies of the machine.

The machine database and the model database are linked with associated rules for predictive analysis. Feedback obtained from a user is utilized to optimize the models even further, thus making them more accurate with time.

FIG. 3 is a flowchart of predictive maintenance analysis using a feedback mechanism.

Referring to FIG. 3, this method aspect of the invention commences with selection of an optimized model for the selected machine type of interest. Once the particular machine type of interest has been identified, data are extracted from the machine database for that particular type of machine and data are extracted from the model database for the selected statistical model for the particular subassembly of the machine of interest. This combination of the data from the two databases is used for predictive maintenance analysis purposes.

The predictive maintenance analysis is performed by the algorithm, and alerts based on the predictive maintenance analysis are sent to the user. The user receives the alerts on a mobile application such as a smart cellphone or a tablet and sends feedback to the cloud where the algorithm and data are stored. The feedback received is used to optimize the training model for further improving its efficiency and accuracy. The process of data transmission from the machine to the cloud is presented in FIG. 1.

Referring to FIG. 1 depicting data flow in the method and system for selecting and updating models for various machines, assemblies, and subassemblies, a pump 100 is illustrated schematically. Affixed to or at least operatively connected to a pump 100 are one or more, and desirably a substantial plurality, of sensors 102 for physical parameters such as vibration, vacuum level, power factor, temperature, relative humidity, voltage, current, and the like. Some or all of sensors 102 are physically connected to pump 100, desirably by mounting thereon, with each sensor being mounted at or on a selected position on pump 100 so as to sense the particular physical parameter of interest at the selected location for that particular sensor.

For example, a sensor 102 for vibration might be mounted on the housing for the pump motor or directly on the motor itself. In the case of vibration, there are several parameters of vibration, not just a single one, that would be of interest and could be used in the model. For example, when vibration amplitude is measured, there is a whole series of harmonics developed from that amplitude measurement. Some of those harmonics may be of interest with respect to particular aspects of vibration; others of those harmonics may be of no interest whatsoever. It is within the scope of the invention to select just certain ones of those harmonics, for example, as the parameter or parameters to be analyzed as respecting the validity of the model and the prediction of machine failure.

A sensor 102 for a vacuum might be mounted on the suction side of pump 100. A sensor 102 for power factor might be wired into the electrical power line connected to pump 100.

Data from sensors 102 is transmitted to a datahub, as indicated by block 2 in FIG. 1. The data transmission is desirably effectuated, wirelessly, preferably using BLUETOOTH low-energy transmission, sometimes abbreviated as “BLE”. Other suitable wireless protocols may also be used; however, BLUETOOTH is preferable.

Data from sensors 102 transmitted via BLE or some other suitable wireless protocol are stored temporarily in a datahub 104, as indicated by block 2 in FIG. 1. The sensor data from datahub 104 are then periodically transmitted from datahub 104 to a router as indicated by block 3, where the router has been designated 106 in the drawings. The router in turn transmits the data wirelessly, desirably over the Internet, to a cloud-resident database 108 as indicated by block 4 in FIG. 1.

A suitable computing device, not illustrated in FIG. 1, communicates with the sensor data resident in database 108 and executes a selected physics and statistically-based mathematical model algorithm, which has been universally validated for particular pumps 100 of interest. A user 110 monitors operation of pump 100 from afar, preferably using a mobile electronic device such as a cellular telephone or a tablet or other personal electronic device, as respecting satisfactory or unsatisfactory operation of the pump.

Still preferably using the mobile electronic device, the user observer sends feedback data to a suitable router which in turn forwards that data to a database 108 resident in the cloud. If the user's information as regarding pump operation was negative, for example if the pump had slowed to an unacceptable speed, an algorithm associated with the cloud-resident data reacts to this feedback information and runs accordingly, updating and if needed, changing the selected mathematical model for pump 100, all as indicated by block 8.

The existing model, and thereby the analysis by the algorithm, is then updated according to the latest operating criteria for pump 100, as indicated by block 9 in FIG. 1.

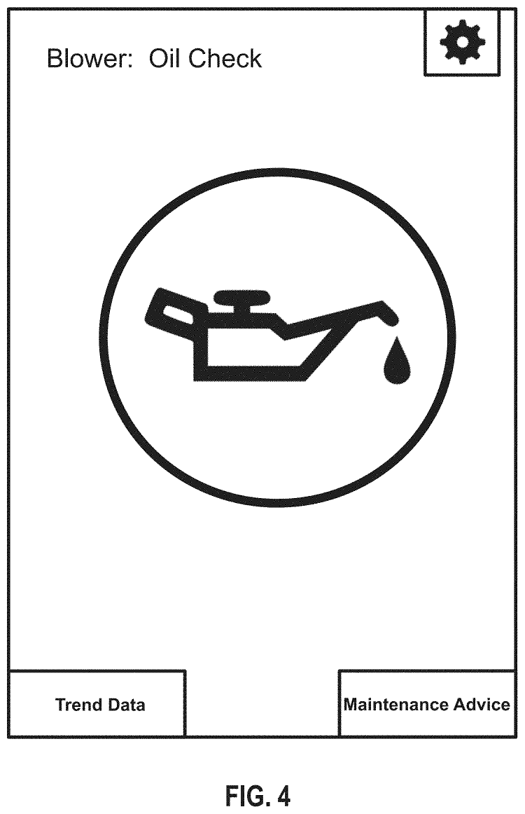

Referring to FIG. 3 depicting data flow in the method and system performing predictive maintenance analysis, a model database 200 contains a list of highly-efficient physics and statistics-based mathematical models for a variety of mechanical, electro mechanical and electrical devices, all of whose models have been universally validated. A machine database designated 202 in FIG. 4 houses data for known machines of different and varying types such as vacuum pumps, blowers, pneumatic dryers, transformers, power rectifiers, three-phase electric motors, single-phase electric motors, and the like.

Predictive maintenance analysis according to the invention and using feedback proceeds initially for a particular machine for which data are available in machine database 202 by selecting an optimized model from model database 200 for the particular machine selected from machine database 202. This optimization and selection may be performed by a user or, more desirably, performed by a selection algorithm based on historical correlation as among machines and models in the databases 200, 202. Once the machine and model have been selected and paired, with the selected machine being assigned to the selected model as indicated in box 4 in FIG. 3, predictive maintenance analysis proceeds for that machine/model combination as indicated in circle 5 in FIG. 3.

Optionally, an observer, preferably using a handheld portable electronic device 112, which may be a cell phone, a tablet, or other portable personal electronic device, may check the pairing of the model and the machine in the course of, or prior to, performance of the predictive maintenance analysis. Data required for the predictive maintenance analysis, namely sensor data collected from one or more sensors 102, sensing one or more physical parameters, which data has been stored in a suitable cloud-resident database 108, is drawn from the database and the predictive maintenance analysis proceeds with a suitable electronic device using that data.

In the case of the exemplary pump analysis as set forth above, and as shown in FIG. 3, sensors for vibration, vacuum, and power factor, for example are connected to pump 100. Data from these sensors is transmitted via BLE to a datahub 104 as indicated by blocks 8 and 9. The sensor data then is transmitted from datahub 104 to router 106 as indicated in block 10, which then forwards the data to the cloud-resident database 108 as indicated by block 11 in FIG. 3.

A user checks the results of the predictive maintenance analysis and provides feedback as respecting the model and the suitability of the model for use with the particular machine in the model-machine pairing used for the predictive maintenance analysis. Desirably, the user is an observer and observes operation of the machine and provides the feedback based on the observed operation of the machine. The user may find the predictive maintenance analysis to be faulty in that the machine may obviously be malfunctioning or not working. At that point, the user provides feedback, preferably using the portable electronic device, most preferably a cell phone or a tablet, connected to a router to transmit the negative result of the analysis to the cloud for a repeat, with either the same data or new data taken from the machine.

In every case, the model utilizes six sigma separation between good data and bad data and good operating state of the machine and a bad operating state of the machine to maximize the accuracy of the analysis.

While the examples provided herein are straight forward and involve only a single sensor and a single parameter of the physical property sensed by the sensor, it is to be understood that the invention may be practiced with multiple parameters, with multiple algorithms, and with multiple series of data taken from multiple different sensors sensing multiple different parameters. Use of the six sigma separation criterion, for the time series or time sensitive data being mined from the sensors, assures high accuracy in the model and failure analyses of this invention.

### EXAMPLE 1

To further illustrate the invention and the method of predictive analysis in one embodiment of the invention, a bearing failure in a blower such as a fan or other piece of turbomachinery may occur.

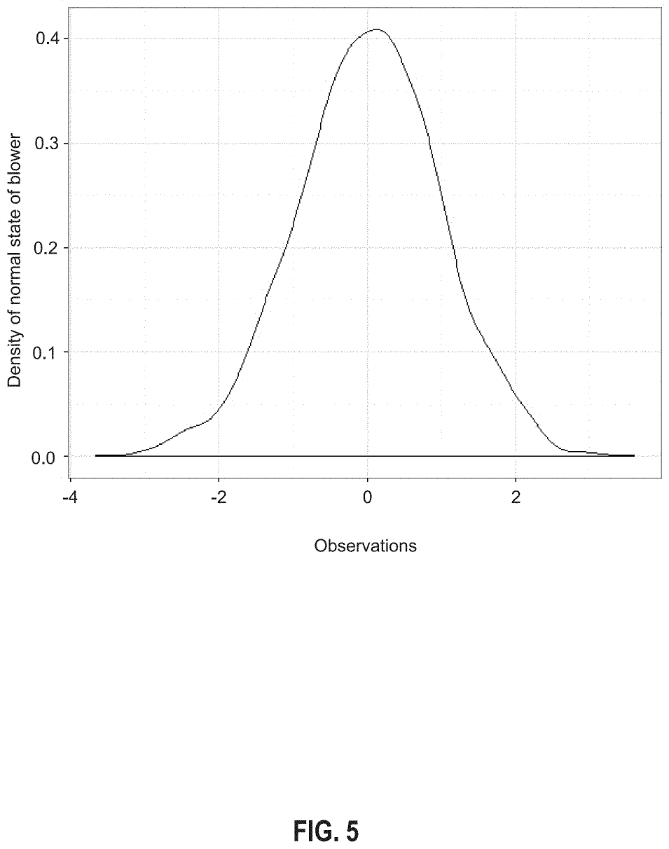

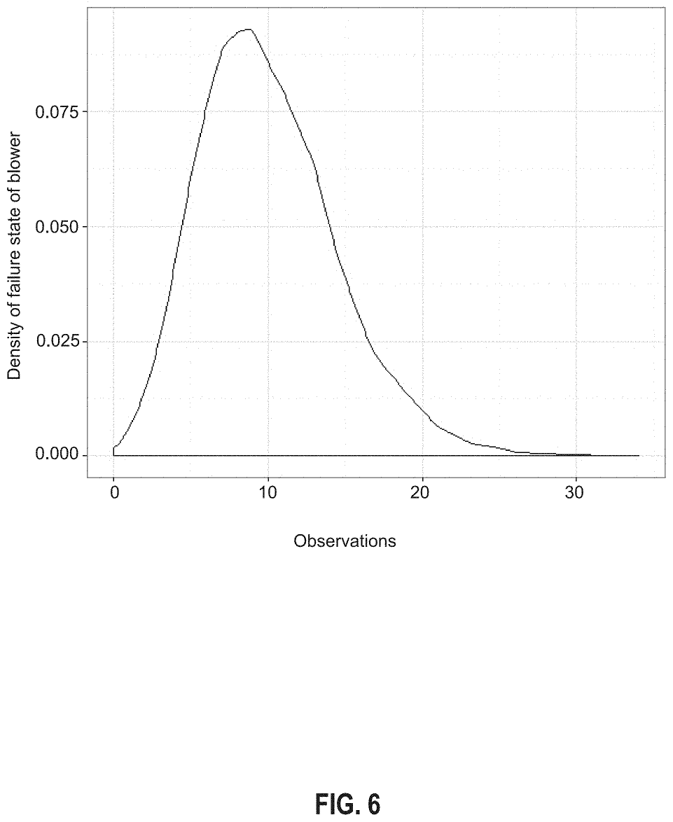

According to a physics based model, when a blower is operating properly, rotating in a plane, the statistical distribution of amplitude or phase of the vibration is normal as illustrated in FIG. 5. On the other hand, when the blower bearing fails, resulting in the rotating fan, shaft, or other piece of turbomachinery moving in a pattern substantially different from the uniform free rotation occurring during normal operation, the distribution pattern of the blower vibration tends to be positively skewed, as illustrated in FIG. 6. This deviation from the symmetrical state is used to predict the normal and failure states for the blower.

When a sensor is deployed towards the end of life of the blower, for example 3 or 4 years after being manufactured, the skewness of the vibration curve may not be exactly zero; rather it may be 0.5 or above. In such a case, the origin is shifted from 0 to 0.5 and any deviation from 0.5 is predicted as the failure state of the blower. This specific information that there has been a shift of origin of 0.5 can only be assessed via a feedback algorithm and presented in the example as the amount of shift that will be automatically discovered based on feedback.

### EXAMPLE 2

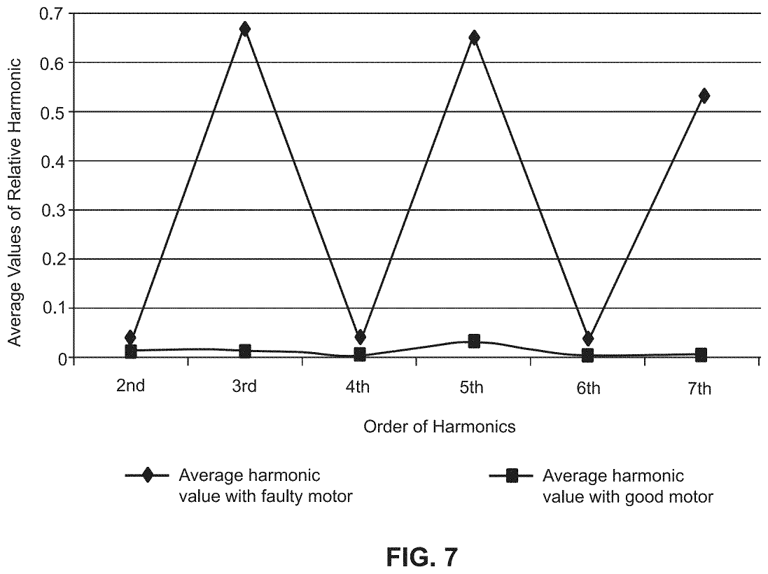

According to a physics based model, when a motor is operating in a good state, the odd order relative harmonics d (3^{rd}, 5^{th} and 7^{th}) of the rotor or output shaft have small values. On the other hand, when the motor fails or approaches a state of failure, these relative harmonics increase in value, reflecting the failure or near failure state of the motor. This deviation of relative harmonics is used to predict the normal and failure status of the motor as illustrated in FIG. 7. Hence a simple threshold, such as 0.3 for example, helps to determine whether the motor has failed or is near failure.

However, when the sensor is deployed towards the end of the motor lifetime, for example 3 to 4 years after the model manufacturing date, the harmonics may not have small values. Hence the threshold of 0.3 needs to be modified based on the then current condition of the motor. In such case, the threshold may need to be shifted to some higher value, such as 0.5. But this specific information that the threshold has a shift of 0.2 from 0.3 to 0.5, can only be assessed via a feedback based algorithm; the amount of shift is auto-determined by the algorithm and the algorithm adjusts automatically to the then current condition of the motor.

While the invention has been described in terms such that one of skill in the art can practice the invention, it is to be understood that the invention is not limited to the description and examples as set forth above. Indeed, other apparatus, methods, and systems, not disclosed herein but which perform substantially the same function in substantially the same way to achieve substantially the same result, are within the scope of the invention and therefore within the scope of the appended claims.

In the claims appended hereto, the term “comprising” is to be interpreted as meaning “including, but not limited to”, while the phrase “consisting of” is to be interpreted to mean “having only and no more”, and the phrase “consisting essentially of” is to be interpreted to mean “the recited claim elements and those others that do not materially affect the basic and novel characteristics of the claimed invention.

A method for maintaining and updating a machine maintenance tool having a physics and statistics based parametric mathematical model of a machine subassembly of interest, which when executed provides parameter readings indicative of the state of machine operation, comprising
a) selecting one or more physical parameters of interest that are a part of the model;
b) connecting sensors for the selected parameters to an embodiment of the machine;
c) using a portable electronic device collecting time series of data from the sensors during machine operation;
d) using a portable electronic device to transfer the collected time series of data to a cloud-based database;
e) executing a physics and statistics based universally validated model for the subassembly of interest using the collected time series data to produce an output of parameter values indicative of the machine condition;
f) if the result is an acceptable improvement on the physical parametric mathematical model of the machine subassembly of interest, replacing the model according to the parameter values reflecting the improvement;
g) if the result is unacceptable, modifying the model and repeating steps “c” through “f”.

The method described in paragraph [0079] further comprising providing a cloud-resident learning engine in operative communication with the database.

The method described in paragraph [0080], wherein the step of executing the physics and statistics based model is performed in the cloud by a big data server operatively connected to the database.

The method described in paragraph [0079] further comprising providing feedback as to whether the result is an acceptable/unacceptable improvement of the model.

The method of described in paragraph [0079] wherein whether the result is an acceptable/unacceptable improvement is provided to an observer; and the observer decides, using the portable electronic device, whether to provide the result of being an acceptable/unacceptable improvement to the learning engine for updating of the model thereby using the feedback data; the observer using the portable electronic device.

The method described in paragraph [0079], wherein whether the result is an acceptable/unacceptable improvement is provided to the learning engine, the learning engine decides whether to update the model using the feedback data.

The method described in paragraph [0079], wherein the parameters have characteristics selected from the group comprising amplitude, frequency, relative humidity, velocity, revolutions per minute, skewness/eccentricity of a rotating member, voltage, current, phase, inductance, impedance, capacitance, surface temperature, infrared temperature, air temperature, and the like.

The method of described in paragraph [0079], wherein the database is a Cassandra.

The method described in paragraph [0079], wherein the data collecting is performed by executing Apache Spark.

A machine maintenance tool comprising:
a) a collection of sensors for sensing parameters having characteristics selected from the group comprising amplitude, frequency, relative humidity, velocity, revolutions per minute, skewness/eccentricity of a rotating member, voltage, current, phase, inductance, impedance, capacitance, surface temperature, infrared temperature, and air temperature, the sensors being operatively connected to the machine by physical mounting thereon or by electrical connection thereto;
b) a router for connecting time series data received from the sensors to a cloud-resident database;
c) a cloud-resident server operatively connected to the database for executing physics and mathematical based models using the time series of data and producing parametric based result data indicative of a machine operating state;
d) a learning engine for executing an auto-correction algorithm using the result data as feedback.

The maintenance tool described in paragraph [0088] further comprising
a) a collection of algorithms allocated to a specific set of models for the machine, which upon receiving feedback from an observer, extend the model by additional statistical models optimized from characteristics extracted by the server from the sensor time series data.

An apparatus for maintaining a machine subassembly, comprising:
a) a collection of sensors for sensing selected physical parameters, the sensors adapted to be operatively connected to the subassembly of interest;
b) a Cassandra database resident in the cloud;
c) a portable electronic device for collecting time series data from the sensors during machine operation and transferring the collected time series data to the Cassandra cloud-resident database;
d) a cloud resident server connected to the database, for executing a physics and statistics based universally validated model for the subassembly of interest using the collected time series data to produce a result, namely whether the database is providing valid data;
e) an electronic device for communicating the result produced by the server to a ground-based observer.

A method of predicting machine failure comprising the steps of:
a) selecting a subassembly of the machine;
b) selecting a universally validated physics and statistically based mathematical model of the selected subassembly;
c) selecting at least one physical parameter from the model for analysis as respecting machine failure;
d) connecting at least one sensor for the selected physical parameter(s) with the selected subassembly;
e) collecting time series data from the sensors at two different times during machine operation;
f) extracting data points for one or more characteristics of the selected physical parameter(s) from the collected time series data;
g) determining whether the extremes of the extracted data points for the selected characteristic(s) of the physical parameter(s) are separated by a preselected criterion; and
h) executing an algorithm processing the extracted data points from one extreme to predict machine failure if the extremes of the extracted data points for the selected characteristic are separated by at least the preselected criterion.

The method described in paragraph [0091] wherein collecting data from the sensor during machine operation further comprises dynamically transmitting the data to a data hub and thereafter transferring collected data from the hub to a cloud resident database for storage therein.

The method described in paragraph [0091] wherein the preselected separation criterion is six sigma.

The method described in paragraph [0091] further comprising checking the predicted machine failure results and if unsatisfactory providing indicia thereof to the cloud resident database for use in updating the selected model.

The method described in paragraph [0094] further comprising detecting the indicia of unsatisfactory results by using a mobile application device and transmitting results of such detection to the cloud resident database.

The method described in paragraph [0091] wherein the physical parameters are selected from the group comprising amplitude, frequency, relative humidity, velocity, revolutions per minute, skewness/eccentricity of a rotating member, voltage, current, phase, inductance, impedance, capacitance, surface temperature, infrared temperature, air temperature, and the like.

The following is claimed:

1. A method for maintaining and updating a machine maintenance tool having a physics and statistics based parametric mathematical model of a machine subassembly of interest, which when executed provides parameter readings indicative of the state of machine operation, comprising:
a) selecting one or more physical parameters of interest that are a part of the model;
b) connecting sensors for the selected parameters to an embodiment of the machine;
c) using a portable electronic device collecting time series of data from the sensors during machine operation;
d) using a portable electronic device to transfer the collected time series of data to a cloud-based database;
e) executing a physics and statistics based universally validated model for the subassembly of interest using the collected time series data to produce an output of parameter values indicative of the machine condition;
f) if the result is an acceptable improvement on the physical parametric mathematical model of the machine subassembly of interest, replacing the model according to the parameter values reflecting the improvement;
g) if the result is unacceptable, modifying the model and repeating steps “c” through “f”.

2. The method of claim 1, further comprising providing a cloud-resident learning engine in operative communication with the database.

3. The method of claim 2, wherein the step of executing the physics and statistics based model is performed in the cloud by a big data server operatively connected to the database.

4. The method of claim 1, further comprising providing feedback as to whether the result is an acceptable/unacceptable improvement of the model.

5. The method of claim 1, wherein whether the result is an acceptable/unacceptable improvement is provided to an observer; and the observer decides, using the portable electronic device, whether to provide the result of being an acceptable/unacceptable improvement to the learning engine for updating of the model thereby using the feedback data; the observer using the portable electronic device.

6. The method of claim 1, wherein whether the result is an acceptable/unacceptable improvement is provided to the learning engine, the learning engine decides whether to update the model using the feedback data.

7. The method of claim 1, wherein the parameters have characteristics selected from the group comprising amplitude, frequency, relative humidity, velocity, revolutions per minute, skewness/eccentricity of a rotating member, voltage, current, phase, inductance, impedance, capacitance, surface temperature, infrared temperature, air temperature, and the like.

8. The method of claim 1, wherein the database is a Cassandra.

9. The method of claim 1, wherein the data collecting is performed by executing Apache Spark.

10. A machine maintenance tool comprising:
a) a collection of sensors for sensing parameters having characteristics selected from the group comprising amplitude, frequency, relative humidity, velocity, revolutions per minute, skewness/eccentricity of a rotating member, voltage, current, phase, inductance, impedance, capacitance, surface temperature, infrared temperature, and air temperature, the sensors being operatively connected to the machine by physical mounting thereon or by electrical connection thereto;
b) a router for connecting time series data received from the sensors to a cloud-resident database;
c) a cloud-resident server operatively connected to the database for executing physics and mathematical based models using the time series of data and producing parametric based result data indicative of a machine operating state;
d) a learning engine for executing an auto-correction algorithm using the result data as feedback.

11. The maintenance tool of claim 10, further comprising
a) a collection of algorithms allocated to a specific set of models for the machine, which upon receiving feedback from an observer, extend the model by additional statistical models optimized from characteristics extracted by the server from the sensor time series data.

12. An apparatus for maintaining a machine subassembly, comprising:
a) a collection of sensors for sensing selected physical parameters, the sensors adapted to be operatively connected to the subassembly of interest;
b) a Cassandra database resident in the cloud;
c) a portable electronic device for collecting time series data from the sensors during machine operation and transferring the collected time series data to the Cassandra cloud-resident database;
d) a cloud resident server connected to the database, for executing a physics and statistics based universally validated model for the subassembly of interest using the collected time series data to produce a result, namely whether the database is providing valid data;
e) an electronic device for communicating the result produced by the server to a ground-based observer.

13. A method of predicting machine failure comprising the steps of:
a) selecting a subassembly of the machine;
b) selecting a universally validated physics and statistically based mathematical model of the selected subassembly;
c) selecting at least one physical parameter from the model for analysis as respecting machine failure;
d) connecting at least one sensor for the selected physical parameter(s) with the selected subassembly;
e) collecting time series data from the sensors at two different times during machine operation;
f) extracting data points for one or more characteristics of the selected physical parameter(s) from the collected time series data;
g) determining whether the extremes of the extracted data points for the selected characteristic(s) of the physical parameter(s) are separated by a preselected criterion; and
h) executing an algorithm processing the extracted data points from one extreme to predict machine failure if the extremes of the extracted data points for the selected characteristic are separated by at least the preselected criterion.

14. The method of claim 13, wherein collecting data from the sensor during machine operation further comprises dynamically transmitting the data to a data hub and thereafter transferring collected data from the hub to a cloud resident database for storage therein.

15. The method of claim 13, wherein the preselected separation criterion is six sigma.

16. The method of claim 13, further comprising checking the predicted machine failure results and if unsatisfactory providing indicia thereof to the cloud resident database for use in updating the selected model.

17. The method of claim 16, further comprising detecting the indicia of unsatisfactory results by using a mobile application device and transmitting results of such detection to the cloud resident database.

18. The method of claim 13, wherein the physical parameters are selected from the group comprising amplitude, frequency, relative humidity, velocity, revolutions per minute, skewness/eccentricity of a rotating member, voltage, current, phase, inductance, impedance, capacitance, surface temperature, infrared temperature, air temperature, and the like.
